(19) United States
(12) Patent Application Publication
(10) Pub. No.: US 2006/0287608 A1
(43) Pub. Date: Dec. 21, 2006

(54) Electromyograph for the detection of electromyographic signals on moving subjects

(76) Inventor: Alberto Dellacorna, Cisliano (Milano) (IT)

Correspondence Address: YOUNG & THOMPSON, 745 SOUTH 23RD STREET, 2ND FLOOR, ARLINGTON, VA, 22202, US

(21) Appl. No.: 11/452,248

(22) Filed: Jun. 14, 2006

(30) Foreign Application Priority Data
Jun. 15, 2005 (IT) MI2005A001129

(51) Int. Cl. A61B 5/04

(52) U.S. Cl. 600/546

(57) ABSTRACT

An apparatus for the detection of electromyographic signals in a moving subject includes signal sensors (S) and movement sensors (F) applied to the subject, each including elements (1) for the analogical picking-up of signals, an amplifier (A) of the detected analogical signals, an analogical/digital converter (A/D) of the amplified signals, a low-consumption microcontroller (2) with independent power supply, for controlling the flow of digital signals to a radio-transmitter (3) apt to sequentially issue the digital signals to a basic unit (U). The basic unit (U) includes a radio-receiver apt to receive the signals coming from the signal sensors (S) and movement sensors (F), a digital/analogical converter, a microcontroller (MCU) and a USB-standard, serial interface unit, the unit thereby being apt to transmit the signals, in digital and/or analogical format, to external devices for signal interpretation and display.

### BACKGROUND OF THE INVENTION

1. Field of the Invention

The present invention refers to an improved electromyograph, particularly suited to detect electromyographic signals on moving objects, such as in particular a human or another animal.

As known, electromyographs are devices which allow to detect the electric activity of the muscles. This detection action may be carried out in static conditions, i.e. with the patient holding still providing to the voluntary contraction of the muscle or muscles of interest only, or in dynamic conditions, i.e. while the patient walks or performs specific trunk, arm or head movements, for the purpose of studying the patient's muscle response during motor activities simulating the ones that the patient performs in ordinary life.

Dynamic electromyography is an extremely interesting and innovative medical technology, since it allows to assess muscle function in actual working conditions and it consequently enables the physician to assess with far greater accuracy and ease the critical steps of the patient's movements and hence to intervene in a targeted manner on the same. Dynamic electromyography is further particularly of interest also in the subsequent therapeutic step, because it allows to thoroughly assess the correctness and effectiveness of rehabilitation actions and therapies performed on the patient and it can also further provide the same with an immediate feedback on the effectiveness of his or her movements, for example during rehabilitation exercises, making learning thereof much faster and satisfying.

Unfortunately, despite these many advantages, so far dynamic electromyography has not met with a satisfactory development due to the poor effectiveness of the electromyographs currently most widely available on the market, which substantially consist of a plurality of electrodes to be fitted to the patient's skin, in correspondence of the muscles, whose activity must be recorded, of a fixed basic processing unit and of a series of electric cables which connect said electrodes to the fixed unit.

This type of electromyographs has been producing satisfactory results in static electromyography, but is affected by various drawbacks when used in dynamic electromyography; as a matter of fact, cable presence is a serious impediment to the patient—who is consequently unable to perform free movements, especially whenever they require considerable displacements—and makes the initial preparation of the patient much more cumbersome. Moreover, electric cable presence and displacement thereof with respect to the patient causes a worsening of the signal/noise ratio, leading to a less exact analysis.

2. Description of the Prior Art

For this reason, electromyographs have initially been provided wherein connection cables no longer consist of wires, but rather of optical fibre bundles, which are much lighter and more compact, and of course free from problems of electrical noise generation. Subsequently, with the object of making patient movement even more free and natural, systems have been provided wherein any patient-to-stationary-unit cable connection has been eliminated and the individual electrodes are cable-connected to a transmitter unit, which unit is sufficiently small to be carried by the patient, said portable unit being suitable to radio-transmit said signals to the stationary basic unit. Lastly, thanks to the recent rapid development of miniaturised electronic technology, systems have been suggested —which are not available on the market yet—wherein each individual sensor is equipped with a miniaturised processing unit and with an own source of energy, so as to be able to communicate directly with the stationary basic unit by means of high-frequency radio waves according to synchronisation and information-exchange protocols, known per se. Moreover, the processing unit of each individual sensor also provides to a first amplification of the electromyographic signal detected by said sensor and to the analogical/digital conversion thereof, so as to minimise the electric noise superimposed on the signal. A system of this type is described in U.S. Pat. No. 6,643,541 and represents the closest prior art of the present invention.

As said above, the electromyograph disclosed in U.S. Pat. No. 6,643,541 has not been effectively introduced in the market yet, and this is probably due to the fact that, as described in the patent, some important implementations appear to be lacking which are essential to allow the practical application thereof in the field of dynamic electromyography. Such implementations hence form the subject matter of the present invention.

In particular, an object of the present invention is to provide an improved electromyograph, by which it is possible to integrate the electromyographic signals with functional information concerning the patient's motor activity, so as to be able to create an exact correlation between the individual steps of each specific patient movement or action and the corresponding muscle activity detected by the sensors.

Another important object of the present invention is to provide an electromyograph, the sensors of which have an extremely simple structure, so as to make the patient-preparation step very short, the length of which is critical for a satisfactory performance of the subsequent test, especially in the case of very young patients.

Another object of the present invention is to provide an electromyograph wherein the sensors, which are put in contact with the patient, consist of sealed containers, so as to guarantee a high standard of safety also when used on children, as well as ease of handling and of sterilisation with disinfectant liquids.

A further object of the present invention is to provide a highly independent electromyograph which is substantially free from disposal problems of the exhaust products, so as to positively meet the demands of continuous operation in a hospital environment as well as the ecological requirements which often represent an important selection parameter in the choice of a device.

### SUMMARY OF THE INVENTION

All such objects are achieved, according to the present invention, through an electromyograph for the detection of bioelectric signals in a moving subject comprising:
a. one or more signal sensors (S) applied to the subject, each comprising means (1) for the analogical picking up of bio-electric signals, an amplifier (A) of the detected analogical signals, an analogical/digital converter (A/D) of the amplified signals, a low-consumption microcontroller (2) with independent power supply, for controlling the flow of digital signals to a radio-transmitter (3) apt to sequentially issue the digital signals to a basic unit (U);
b. one or more movement sensors (F) applied to the subject, each comprising means for the detection of a patient's motor activity in the form of an analogical electric signal of varying intensity, an amplifier (A) of the detected analogical signals, an analogical/digital converter (A/D) of the amplified signals, a low-consumption microcontroller (2) with independent power supply, for controlling the flow of digital signals to a radio-transmitter (3) apt to sequentially send the digital signals to said basic unit (U);
c. at least a basic unit (U), comprising a radio-receiver capable of receiving the signals coming from said signal sensors (S) and from said movement sensors (F), a digital/analogical converter (D/A), a microprocessor (MCU) and a USB-standard, serial interface unit, said basic unit being capable of transmitting said signals, in digital and/or analogical format, to external devices for signal interpretation and display.

According to a feature of the invention each sensor (S, F) consists of a closed and sealed element, containing the processing components of the detected signal and a means for the independent power supply of the microcontroller (2), whereto said means for the picking up of the bio-electrical signals or said means for the detection of a motor activity are electrically and mechanically connected. The means for the independent power supply of the microcontroller (2) consists of a rechargeable battery (5), permanently enclosed in said closed and sealed element.

According to another feature of the invention each sensor (S) is completely insulated from the environment and is further miniaturised so as to make the difference between the common-mode voltages existing on the electrodes (1) and on the amplifier (A) negligible; thanks to this configuration, said picking-up means consist of the two signal-detection electrodes only, the sensor (S) hence lacking a third equalisation electrode.

According to a further feature of the invention, the basic unit (U) comprises a radio-transmitter capable of sending digital signals to the sensors (S, F) for controlling the power consumption of the battery (5), through the configuration of said sensors (S, F) into four different activation states comprising: a low-consumption SLEEP state, a transient synchronisation SYNC state, a waiting READY state, and a data acquisition OPERATE state.

According to a still another feature of the invention, each electrode (S, F) further comprises a plurality of buffer memories (BM), used sequentially for storing the packet of data issued to basic unit U for which basic unit U has not sent a correct reception signal to the sensor (S, F), said packets of data stored in such buffer memories (BM) being transmitted back to the basic unit (U), at the end of the acquisition session of the patient's data, up until correct reception of the same.

### BRIEF DESCRIPTION OF THE DRAWINGS

The electromyograph according to the present invention will now be better described with reference to some preferred embodiments of the same, detailed in the following, also in connection with the accompanying drawings, wherein:

FIG. 1 is a diagrammatical general view of the improved electromyograph according to the present invention;

FIG. 2 is a block diagram which shows the components of a central basic unit of the electromyograph;

FIG. 3 is a block diagram which illustrates the components of an electromyograph sensor;

FIG. 4 is a top plan diagrammatical view of a battery “docking”, i.e. a container apt to house and recharge the sensor batteries;

FIG. 5 is a wiring diagram which shows the electrical generator circuit of two adjacent positions of the docking and the receiving circuit of two sensors inserted in the docking;

FIG. 6 is a wiring diagram which illustrates a detection circuit of the electromyographic signal in a sensor according to the currently used prior art; and

FIG. 7 is a wiring diagram which shows a detection circuit of the electromyographic signal in a sensor according to the present invention.

### DESCRIPTION OF THE PREFERRED EMBODIMENTS

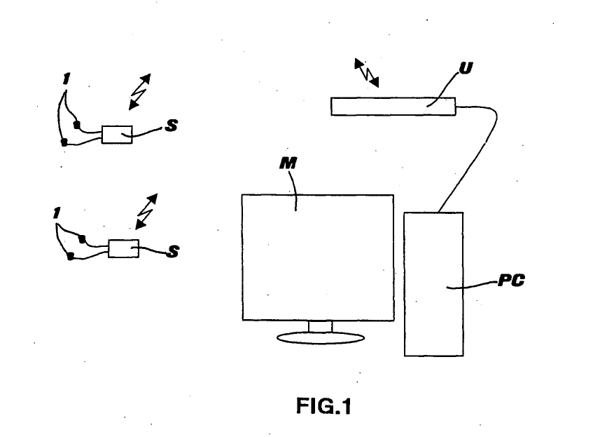

As shown in FIG. 1, the electromyograph according to the present invention comprises a fixed basic unit U and one or more sets of wireless sensors S, each set preferably consisting of eight differential sensors. Basic unit U is operated both manually and by a main (host) processor PC, connected with unit U through a USB interface and equipped with a screen M for the immediate display of data and information. Through a suitable software installed on the processor PC, it is then possible to programme the various functions of unit U and of sensors S.

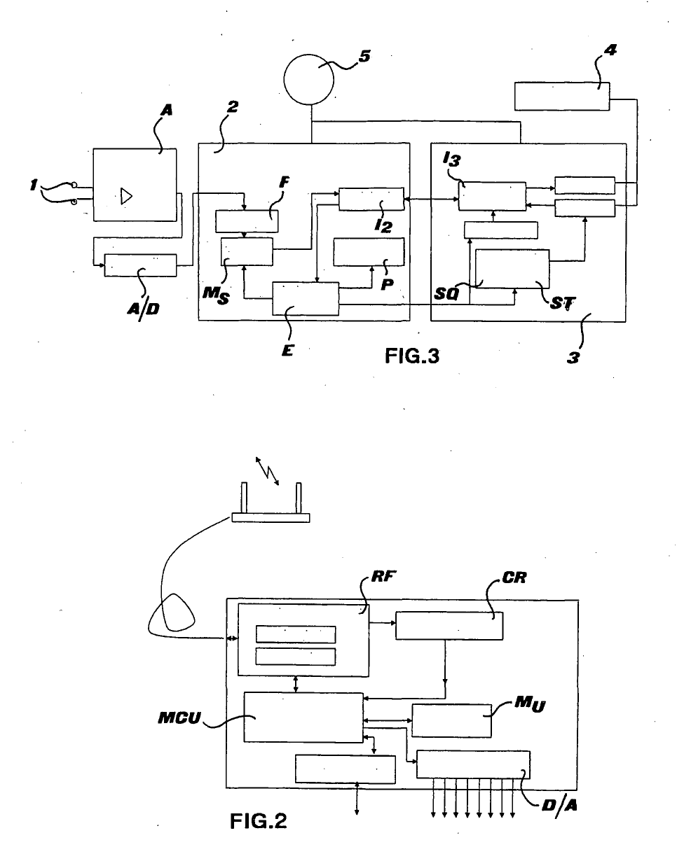

In FIG. 2 it can be appreciated that basic unit U comprises a 2.4 GHz receiver-transmitter module RF with the relevant antenna, a microprocessor MCU, a digital/analogical multiple converter D/A connected with an analogical output, a receiver switch CR, and finally a USB interface for connection with the processor PC. Unit U is equipped with a universal, 90-240 Vac power supply, for direct connection to an outlet. The system uses a single frequency in the 2400-MHz ISDN bandwidth; the frequency may be programmed via software through the processor PC, to comply with international rules and regulations. The system is capable of operating in environments where a WLAN 802-11 is found.

All the functions of the receiver-transmitter module RF are twofold to allow operation in diversity mode, so as to minimise the effect, on the radio-transmitted signals, of patient movements during the data acquisition step, which phenomenon is known as multi-path fading. This phenomenon, caused by the reflection of radio waves on the objects in the environment, is particularly significant in case radio systems are used indoors, where it causes data loss especially when the source of radio waves (in this case the patient) is moving—as happens in dynamic electromyography.

As a matter of fact, the receiver antenna is affected by the electromagnetic radiation coming from the antenna of the transmitter sensor, but also by the reflected electromagnetic radiation generated by the same transmitting sensor, which has covered paths of varying lengths. Consequently, when the voltages induced on the receiving antenna are mutually phased in, a strengthening of the received signal will be obtained; viceversa, when they are bucking voltages, an attenuation will be the result. This problem is strongly reduced by the application of a second receiver equipped with an antenna of its own. As a matter of fact, the likelihood that critical conditions occur simultaneously on two different antennas is very low, and correct reception of transmitted data is thereby guaranteed.

After having been received by module RF, the digital data containing the electromyographic information are provisionally stored in a buffer memory Mᵤ and hence made available in output both in an analogical format, through converter D/A which is equipped with a standard, D-type output connector, and in a digital format, through the processor PC connected with USB interface.

In the block diagram of FIG. 3, the different components of a sensor S for the detection of electromyographic signals are shown. Each sensor comprises firstly a pair of electrode-carriers 1 connected with the body of sensor S through flexible cables; this solution remarkably simplifies electrode arrangement in the areas most suited to detect the desired signal and further allows to adjust in an optimal manner the mutual distance between the same. Fixed electrodes integral with the body of sensor S may of course nevertheless also be used. Said body consists of a fully enclosed and sealed container, preferably parallelepipedal in shape and having a low thickness, which may be easily fitted to the patient's body by adhesive means known per se. Electrode-carriers 1 are equipped with button clips for the application of the disposable electrodes normally available on the market.

A differential amplifier A receives the analogical electromyographic signal from the above-mentioned pair of electrodes 1, amplifies it and makes it available to a converter A/D which performs the digital conversion thereof. The digital signal is then sent to a low-consumption microcontroller 2, preferably consisting of a single integrated microcircuit (“single-chip”), wherein the signal undergoes digital filtering in filter F and is finally temporarily stored in a buffer memory Mₛ. From memory Mₛ the signal is then taken and transferred to a digital receiving-transmitting interface 3, itself of course operating in a 2.4-GHz bandwidth, which transmits the signals to basic unit U by means of an integral antenna 4 included in the body of sensor S.

Also the receiving-transmitting interface 3 is preferably of the type consisting of a single integrated microcircuit which comprises, in addition to the-proper receiving-transmitting functions of digital signals, also the functions of a sequencer SQ of the data exchanged between microcontroller 2 and interface RF 3 through suitable data interfaces I₂ and I₃, and finally those of a phase-control synthesiser ST for the programmable generation of transmission frequency.

All the functions of sensor S are programmed and controlled by a processing unit E located in microcontroller 2, under the control of basic unit U from which it receives the synchronism as well as the operation and configuration commands. The processing unit E of microcontroller 2 also provides to the management of power resources of sensor S through a supply unit P connected with a rechargeable lithium battery 5. Supply unit P provides to minimise the consumption by sensor 2 during the passive steps of the application to the patient and when the system is not in use.

On the contrary to what is currently normally provided in portable devices, for the purpose of allowing a perfectly sealed construction of sensor 2—thereby increasing safety of use also on children and allowing easy and practical sterilisation thereof—battery 5 is not of the type accessible from outside and hence replaceable by the user, but it is permanently inserted, during manufacture, into the body of sensor S. In order to charge the battery 5 included in each individual sensor S, according to the present invention a special system has been devised which does not require any physical connection with the battery, consequently also removing the usual outer terminals by which rechargeable batteries are normally connected with their respective power supply units.

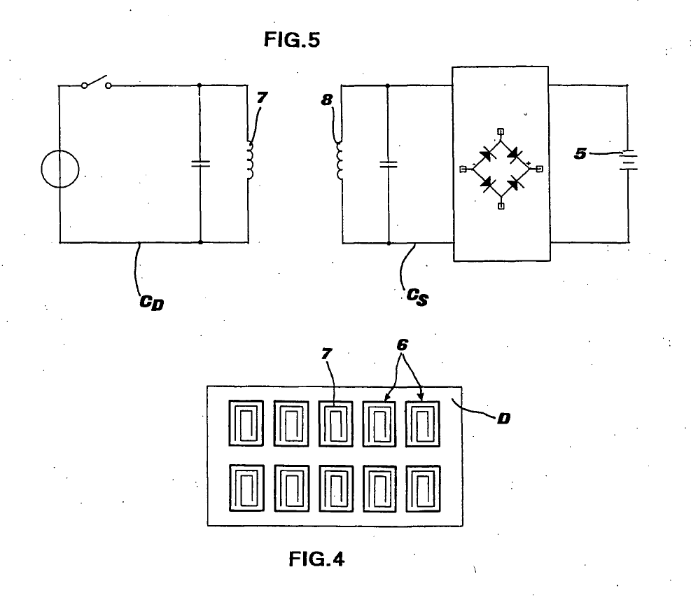

Such system consists of a container D apt to house and recharge sensors S (FIG. 4), called “docking” in short, equipped with multiple housings 6, to allow the simultaneous recharge of the entire set of 8 sensors with respect to a same basic unit U. In each of housings 6, acting as a support base of a sensor S, a respective coil 7 is printed; energy transfer to battery 5 occurs by induction through the mutual coupling of the coil 7 of a circuit C_{D} (FIG. 5) with a corresponding coil 8 provided in the electric recharging circuit C_{S} formed within sensor S.

“Fixed” coils 7, arranged on the docking, are energised to come into resonance by impulses of suitable amplitude and frequency; the “mobile” coils 8 of sensors S—when these are positioned in their respective housings 6 provided on docking D—are consequently run through by an induced alternate current which, following a suitable rectification, is sent to battery 5 for recharging.

Activation of sensors S occurs, as mentioned above, following a radio code issued by basic unit U; by means of this “up-link”, all acquisition functions of sensors S are further programmed. Transmission of the electromyographic data by the active sensors is carried out according to a preset sequence, using a “down-link” channel, each sensor having for this purpose a time window for the transmission of the data relevant to it. The sequential cycle is repeated until the end of the acquisition.

The data string transmitted by sensors S contains, in addition to the electromyographic samples, also the codes required for checking for integrity of the transmitted data and for channel identification. Data frequency during down-link is 1 Mb/sec, and that allows the electrode to remain in stand-by mode, as better explained in the following, for the major part of the cycle, thereby minimising energy demands.

The system may be expanded by using multiple basic units U, by assigning a different frequency to each set of 8 sensors S. Each set of sensors hence operates on its own frequency, regardless of the others. With the prototype system currently devised by the inventor, it is possible to use up to 4 sets of 8 electrodes each, up to a total of 32 points of simultaneous information detection on the patient. For ease of identification, each sensor is preferably marked out by a progressive number ID (from 1 to 8) and by a colour (red, green, blue, yellow) showing the sensor belonging to one of multiple sets of eight sensors.

For the purpose of achieving as low an energy consumption as possible, sensors S are pre-programmed so as to be able to work in four different operation states, called SLEEP, SYNC, READY and OPERATE, and activated by basic unit U.

The SLEEP state is the default inactive state of sensor S, during which its energy consumption is reduced to extremely low values; sensor endurance in the SLEEP state is hence very long, preferably over a year. In this state, microcontroller 2 provides to cyclically activate sensor interface RF 3 in a reception window having a very short duration; for example, in a cycle of 10-180 sec, the duration of the reception window ranges between 1 and 18 msec. In such window, it is checked for the presence of a signal RF coming from basic unit U, which signal contains an activation command of the specific sensor, for transferring the same to a synchronisation state (SYNK).

Sensor S enters the SLEEP state when it receives a SLEEP command from basic unit U (which control is sent by default when unit U is switched off). For the purpose of increasing, where necessary, the useful duration of the full charge of battery 5, it is possible to arrange sensor S, so that it may enter the SLEEP state by timeout, after a preset period of time has elapsed from reception of the last valid code.

In any case, sensor S automatically enters the SLEEP state following a full discharge of battery 5.

The SYNC state is a transient one, required for the synchronisation of all the sensors with basic unit U. Once it has been activated in this state, each sensor S hence performs a longer reception cycle of a duration comprised for example between about 0 and 1 sec, to allow basic unit U to synchronise all the sensors. As a matter of fact, in the SLEEP state each sensor operates in an asynchronous manner with respect to basic unit U and it is hence necessary to first bring all sensors S in synchronism before making the system operational.

Sensor synchronisation occurs according to the following steps:

basic unit U transmits the SYNC command for a length of time equal to the duration of the SLEEP cycle of sensors S;

each sensor receiving the command puts itself in the SYNC state in continuous reception;

as soon as basic unit U detects the switching of a sensor to the SYNC state, it transmits a READY command to such sensor;

the sensor receiving the READY command activates a synchronous cyclic operation with a low-frequency repetition.

At the end of the above-mentioned period of time, all the sensors have hence synchronised with basic unit U. The SYNC state may be activated in a manner transparent to the operator, for example upon switching on of basic unit U.

The READY state is the pre-operational state wherein sensor S performs the sequential transmission cycles under the control and the synchronisation of basic unit U; since the sensor functions are only partly activated, power consumption by the sensor is lower than in the acquisition step. As a matter of fact, the analogical blocks of the sensor are not active in this step; as a result, only the following data are transmitted to basic unit U:

battery voltage;

electrode impedance (to allow the detection of any electrode detachment);

identification code.

In order to preserve the synchronism of the different sensors, it is of course necessary for the READY operation cycle to be repeated with a predefined frequency, taking into account the tolerances of the time base of each sensor; the frequency of cycle repetition is low (for example comprised between 0 and 1/100 of the cycle frequency in the operational step).

The OPERATE state, finally, is the state of acquisition of the electromyographic data, during which sensor S is fully active, under the control of basic unit U. The sensors transmit the electromyographic data to basic unit U at the maximum operational frequency, for example with sampling frequencies comprised in the range from 100 to 10,000 samples/sec.

When the operator demands detection of electromyographic signals, during a READY cycle, basic unit U transmits the OPERATE command and from the following cycle all sensors S begin to transmit the electromyographic data. Simplified versions of the control system of energy consumption by sensors S may also be used, for example whenever the charge of battery 5 is redundant with respect to the power requirement of a full day's work, fully eliminating the intermediate SYNC and READY steps and hence immediately activating the sensors in the OPERATE state as soon as they are woken up from the SLEEP state.

The configuration and control registers of sensor S comprise:

base registers wherein the base configuration of the sensor is effected, by defining the frequency and operating power thereof, the operating mode, etc.;

a key register of the base registers which enables reconfiguration of the receiving-transmitting interface 3 with new data;

operational registers wherein the operating mode of the sensor as well as the activation/deactivation of the various operating steps are defined.

All the registers are stored in a set-up memory; a change of set-up causes a corresponding immediate change of sensor operation. Upon each activation of the electromyographic device of the invention, each sensor S is automatically set according to the set-up it had at the time of the last deactivation. An exception is the case in which the sensor battery is fully discharged; in this case, following battery recharge, the default set-up is automatically enabled.

Communication between sensors S and the basic unit occurs in half-duplex mode, by using a single frequency, chosen among 125 values (2400-2524 MHz). The system can perform, in an automatic manner, short transmission cycles with different frequencies and establish the best frequency on the basis of any detected mistakes. Data transfer is executed in FSK mode with a fixed, 1-Mbps data rate. Transmission occurs packetwise, with a total of 256 bit per packet.

The up-link channel (communication from the basic unit to the sensors) transfers the operational codes and the operation parameters; the down-link channel (communication from the sensors to the basic unit) transfers the data.

Any data transfer on the RF (up-link or down-link) channel implies the transmission of a 256-bit packet, consisting of an ADDRESS, a PAYLOAD, a CRC.

The packet transmitted from basic unit U to sensors S (up-link channel) consists of the following:

ADDR, 32 bits, address of sensors S;

PAYLOAD, 208 bits (26 bytes);

CRC, 16 bits.

The address of the sensors is fixed for each set of 8 sensors; during the up-link, all 8 sensors simultaneously receive the information from the basic unit; if ADDR and CRC are correct, the PAYLOAD is made available to microcontroller 2. The PAYLOAD consists of:

120 bits for sensor configuration (base registers), which determine frequency, output power, data format and anything else necessary for basic sensor configuration. These data are used only if the default setup has to be changed;

16 bits for sensor configuration protection (key of the base registers); it is used to implement any new sensor configuration;

72 bits for functional control of the sensor (nine operational registers); they are used to activate or deactivate the above-defined, four different operational states of the sensor and to define the functional parameters chosen by the user, such as the sampling frequency.

Each sensor S uses the down-link channel only in the following two operational states: SYNC and OPERATE. Transmission on this channel occurs packetwise, with packets consisting of:

ADDR, 32 bits, address of basic unit U;

PAYLOAD, 208 bits, 12 samples of 16-bit+16-auxiliary-bit electromyographic signals;

CRC, 16 bits.

The down-link channel may be used by one sensor at a time only and consequently, in case of multiple sensors, data transmission must be sequential. Each sensor is assigned in production a non-changeable number from 1 to 8, which is associated with a transmission delay which is a multiple of said number. Sensor number 1 is therefore the first to engage the down-link channel, followed by number two, and so on as far as number eight. Data transmission is required simultaneously by the basic unit to all sensors, which provide automatically, according to their own transmission delay, to the transmission of sequential data.

According to a fundamental feature of the present invention, the above-described basic electromyograph is integrated with a second series of sensors F which detect functional information on the patient's motor activity, so as to be able to create an exact correlation between the individual steps of each specific patient movement or action and the corresponding muscle activity detected by sensors S.

The sensors which may be used in a medical environment as an integration of the electromyographic information are of course: accelerometers, to detect the speed with which a given body segment has moved; electrogoniometers, to measure the amplitude of joint displacements; foot switches, for the detection of foot contact with the ground during deambulation and hence to provide an indication of step stages; load cells, for the detection of the weight or of the force imparted by the patient to a resisting member (strain gauges); pressure gauges, for the recording of the patient's blood pressure; temperature gauges, for the recording of the patient's body temperature.

In general, accelerometers and electrogoniometers are analogical sensors; the data recorded by these sensors are hence processed and transmitted in the same way described above in detail for electromyographic signals. Of course, given the different magnitude of the forces at play, sensor F in this case is suited to directly generate a signal of an amplitude suitable to be easily processed (i.e., it is generally not necessary to process the signal with previously-described amplifier A).

The foot switch sensors may be simple switches (two thin laminae which come in contact if pressure is applied thereto) or piezoresistive sensors, i.e. based on special conductive polymers which vary electric conductivity according to the pressure they are subject to. In the former case, the sensor circuits must detect and transmit a simple on/off state, in the latter they must instead detect variations in the electrical resistance of the piezoresistive sensors, which leads again back to the acquisition of an analogical signal. It will then be basic unit U which interprets the signal and delivers the sensor state (loaded or unloaded, as well as load intensity) as output. With a suitable system pre-calibration, the analogical signal thereby received represents the variation over time of the pressure the tested subject applies to the sensor. Systems of this type are available on the market, equipped with hundreds of sensors, which allow for example to acquire the dynamic map of weight distribution during foot resting. Signal interpretation may of course also take place within the sensor itself; in this case the state of sensor F only will be transmitted. In both cases the circuit threshold may be calibrated according to the subject's weight (for example children or adults). It is further possible to implement a function for the automatic definition of the threshold value, on the basis of a system pre-calibration (the subject takes a few steps, the system measures pressure values over time, calculates maximum and minimum values and establishes the threshold value at a suitable intermediate value).

The load cells may be useful to correlate the electrical activity of the muscle to the mechanical force generated during contraction; for example during isokinetic rehabilitation. These sensors generate low-level signals and require a treatment similar to the ones seen previously for electromyographic signals.

The different types of sensors F already present on the market for the detection of functional information on a patient's motor activity, may be integrated in the electromyographic device according to the present invention by simply equipping them with a microcontroller and with RF interfaces fully similar to those mentioned above in connection with sensors S for the detection of electromyographic signals.

It will thereby be possible to provide sets of “miscellaneous' sensors of the S and F type controlled by a single basic unit U, or “pure” sets of S and F sensors controlled by different and respective basic units. In any case, according to the present invention, the system is capable of coordinating and communicating with both types of the above-mentioned sensors, so that the final information supplied to the physician or to the therapist may include both the signals relating to the patient's muscle activity, and the signals relating to the patient's motorial function, without the patient being hindered in any way in such activity by the presence of connection members between the patient and the basic unit or units and the control PC of the same.

A preferred embodiment of the S/F sensors of the electromyograph of the present invention will now be described, which embodiment allows to simplify and remarkably speed up the initial patient preparation step, i.e. arranging said sensors on the patient's body surface, with respect to the reference prior art.

As is well-known to people skilled in the field, the bioelectric signals generated by the muscle—especially in the case of surface electromyography, which is the technique habitually used in dynamic electromyography—are characterised by a very modest amplitude, of the order of a few hundreds of microvolts. It is hence generally necessary, as already seen above, to provide to significant amplification of such signal in order to be able to then have an output signal of a level adequate to following processing operations.

Such amplification is normally effected through differential amplifiers, which accomplish the amplification of the potential difference found between the two inputs they are provided with, which are connected with electrodes 1. An ideal differential amplifier is sensitive only to the difference between the two inputs thereof; the actual differential amplifier is instead sensitive also to common-mode voltages at the two inputs.

Common-mode voltages are those existing between the amplification circuit reference (for example the electric “0” of the circuit) and both conductors connected with the two amplifier inputs, such conductors are hence “run through” by noise currents; these currents, thanks to the circuit symmetry, produce a null differential effect, that is they do not interfere, in a differential sense, with the useful electromyographic signal.

Not negligible is instead the effect due to the non-infinite amplifier attenuation of common-mode rejections; as a result, the less the amplifier is sensitive to common-mode voltages, the better its quality. Manufacturers of differential-amplifier devices state the CMRR (common-mode rejection rate) parameter, which represents the attenuation of the common-mode voltage; with common-mode voltage being the same, the higher the CMRR value, the smaller the influence of common-mode voltage on the useful signal.

These common-mode voltages are always present in experimental set-ups, and may take up amplitudes which are several orders of magnitude greater than the amplitude of the signal to be amplified, when signals having very small amplitude are processed (for example hundreds of microvolts, as in surface electromyography). According to the prior art, in these cases a third “equalisation” conductor is used in order to eliminate the distortion effect of common-mode voltages; moreover, the third conductor serves to bring the potential of the inputs of the differential amplifier within the operational limits established by the manufacturer.

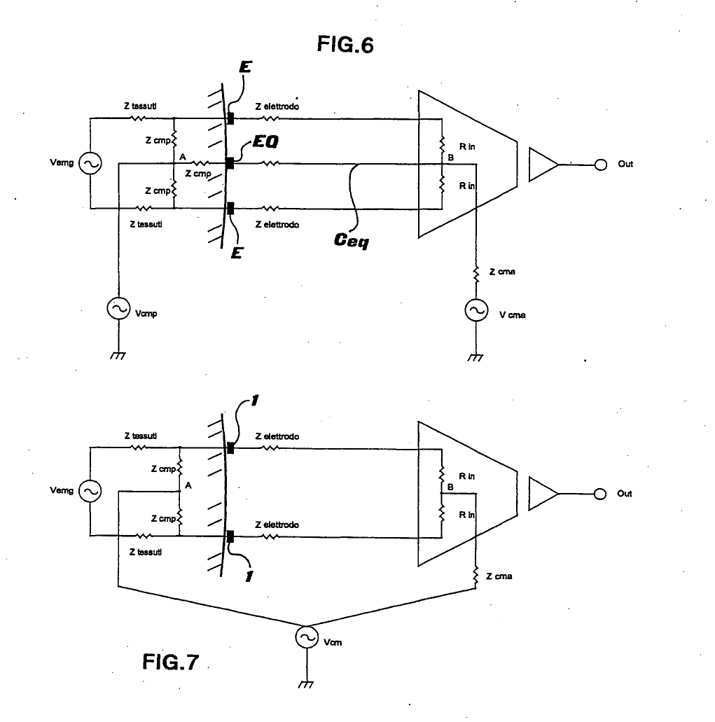

FIG. 6 shows an equivalent diagram of a typical connection between the electrodes acquiring the electromyographic signal and the patient, according to the teachings of the prior art. In the drawing:

Vemg is the electrical potential available at the muscle being tested;

E are surface electrodes, applied to the patient's skin at a short distance from one another;

Ztessuti is the impedance of the tissues found between the muscle and electrodes E;

Zelettrodo is the contact impedance of the electrode;

Rin is the input resistance of amplifier A.

The patient is exposed to the electric fields found in the working environment (for example those generated by conductors of the mains supply) which may generally be represented by a generator Vcmp fitted, by means of an impedance Zcmp, to the picking-up points represented by electrodes E.

The measurement equipment, too, is subject to the electric fields found in the working environment, and this phenomenon may be similarly represented by a generator Vcma, applied to the input circuit at its unipotential point B by means of an impedance Zcma.

It has been stated that, due to the presence of common-mode voltages, the conductors connecting electrodes E with the amplifier are “run through” by noise currents and that such currents, thanks to circuit symmetry, produce a null differential effect. On the contrary, the common-mode voltage which appears to be applied between nodes A and B, i.e. the sum of Vcmp and Vcma, although attenuated due to the CMRR of the amplifying device used, will in any case be present as a noise in the output signal.

The technique currently used in order to minimise this problem consists, as mentioned, in adding a third conductor Ceq for the “equalisation” of the common-mode potentials on nodes A and B, connected with a reference electrode EQ, arranged at the same distance from electrodes E. However, while the application of this technique to conventional electromyographs raises no particular problem—since a single equalisation conductor is sufficient—provided it is suitably fitted to the patient—to eliminate the problem of common-mode voltages of a plurality of sensors, considering that these sensors are all connected with a single device and consequently all have the same potential—the same does not occur in wireless electromyography, where each sensor is set apart from the others and must hence have a specific “third” equalisation electrode of its own. This situation is clearly highlighted in U.S. Pat. No. 6,643,541, which represents the reference prior art of the present invention, wherein each sensor is indeed described as also comprising—in addition to the two electrodes for the detection of electromyographic signals—a third electrode—attached at the end of a suitably long conductor, in order to achieve at least an approximately equal distance from the two detection electrodes—which is connected with the signal amplifier to accomplish the above-described conductor for the equalisation of the common-mode potential.

However, a solution of this type implies a 50% increase in the number of electrodes to be fitted to the patient's body over prior-art electromyographs, with a resulting increased time effort by the operator and increased inconvenience to the patient.

From the extensive studies carried out by the inventor on this subject, and as subsequently confirmed by experimental evidence, it resulted instead that extreme miniaturisation of S/F sensors allows to decrease common-mode voltages to negligible values without using a third reference conductor. This surprising result, fully in contrast with prior art teachings, is due to the fact that, thanks to the extremely reduced sensor size, to their arrangement in strict proximity to the source of the signal and to the coupling via RF which leaves the sensor in an insulated condition, the sensor itself is strongly coupled with the patient's body and very little with the surrounding environment. As a result, the sensor will follow the patient's potential, hardly being affected by the perturbation of the surrounding environment.

This situation is represented in the diagram of FIG. 7, which refers to the operating condition of the sensor according to the present invention, and differs from the diagram of the prior art shown in FIG. 6, precisely for the lack of the equalisation conductor Ceq and for having only one generator of common-mode voltage. As a matter of fact, this condition occurs when the patient and the electrode substantially undergo the same perturbing field—for the reasons set forth above—represented in the diagram by generator Vcm. In this case, the potential difference which develops between nodes A and B due to the perturbing field is evidently null, as are the effects of the environmental electric field on the electromyographic signal which is to be detected.

The above-described theoretical condition is accomplished with optimal approximation in the case of the above invention, thanks to the extremely reduced size of the circuits of the S/F sensor and to the total lack of electrical connections between the reception system, consisting of basic unit U, and the sensor itself.

The innovative solution described above may then be further improved by altering the electric circuit shown in FIG. 7 so as to allow an even greater electrical “equalisation of the potentials” between the patient and the circuits of the S/F sensor; this result may be obtained by applying two same-value resistances between electrodes 1 and the unipotential node B of the amplifier, said resistances being arranged in parallel to the conductors carrying the electromyographic signal. However, this solution is effective only if the introduced resistances have a very high value, such as not to interfere with the differential signal, but significantly lower (at least by the order of 10⁻³) than the input resistances Rin of the amplifier. This may be achieved by adopting latest-generation amplifiers A wherein the resistances Rin have a value of the order of 10¹³ Ohm and hence the additional resistances in parallel may have a value up to 10¹⁰ Ohm, thereby fully achieving the objects of the invention.

As known, data transmission in a digital format implies the possible loss of part of the data. In order to quantify the loss, a parameter is used, the Bit Error Rate (BER), representing the ratio of the number of errors occurred to the total number of transmitted bits.

The BER parameter value mainly depends on the medium used for transmission; in the case of radio transmission, the medium is necessarily shared with any other application, and this implies a high risk of errors and a correspondingly high BER value. The presence of distorting transmissions, which may be assimilated to noise sources, decreases the ability of the receiver to discriminate the useful signal among those present, and this may account for wrong bits in the received data.

In order to eliminate transmission errors, the use of error detection systems is known, which allow to detect the presence of errors in the data received. In known devices, whenever an error is detected in a packet of received data, a new data transmission is requested, and this cycle is reiterated—if necessary—until the packet in question is received undamaged. This technique requires a greater transmission bandwidth, since in addition to the original data, the space must be provided for transmission of corrections; the transmission of the correct packets is further asynchronous with respect to the original data, i.e. it causes a variable and nonforeseeable delay between the instant in which the data are transmitted and the instant in which the (corrected) data are received.

A feature of the electromyograph according to the present invention, particularly interesting in dynamic electromyography applications, is instead that the data coming from the patient are received “in real time”, i.e. that they are available to the receiver with a short delay, which is the same for all data and constant over time, therefor at a frequency equal to the one with which the data have been generated. Owing to the limitations imposed by real-time synchronous transmission, in this case it is not possible to provide data retransmission in case of incorrect reception: incorrect data are hence simply rejected, while the display keeps showing the latest data received correctly. In environments free from particular, radio-electrical disturbances, the level of errors is in any case very low, in the order of one incorrect packet of data every several thousand transmitted ones, so that the approximation introduced in the received data is virtually negligible.

However, according to a peculiar feature of the electromyograph of the present invention, the opportunity is provided—which is useful when the device is used in environments where other radio-transmission devices or other sources of electrical disturbance are found—to implement the electromyograph along with an error detection system, which guarantees on the one hand that the data displayed on screen or in otherwise provided are correct, synchronous and delivered in real time and, on the other hand, that the detected data, regardless of any transmission errors occurred during real-time transmission, are finally completely correct and stored in an archive file for subsequent examination by the physician.

According to such integrative system, it is provided that each electrode S be equipped with “n” buffer memories, taking the place of the above said single memory Mₛ, each capable of storing one data packet. When electrode S transmits a packet of data in “real time”, such data are simultaneously stored in a first buffer memory BM1, selected by the receiver through a corresponding switch SM1.

Once the packet transmission step has ended, the electrode receives from the basic unit the code for synchronism and for the start of the new transmission cycle. A code showing the “correct/incorrect reception” of the previously transmitted packet of data is added to this information. If the previously transmitted packet has been received correctly, sensor S keeps switch “SM1” closed and proceeds to the sending of a new packet of data, simultaneously overwriting it on the same first buffer memory “BM1”, in place of the data of the previous packet. If, on the contrary, the previously transmitted packet has not been received correctly, and has hence been rejected by basic unit U, sensor S opens switch “SM1” and closes second switch “SM2”, thereby selecting a second memory BM2; the buffer memory function is hence now assigned to such second memory “BM2”, whereas memory “BM1” definitively stores the data which were not received correctly in the previous cycle. Whenever basic unit U later signals incorrect reception of a packet of data, such packet of data is hence stored in a subsequent, available memory area BMn of sensor S.

At the end of the signal acquisition session, the packets of data stored in buffer memories BM are sequentially transmitted to basic unit U and if incorrect reception occurs again, the receiver provides to request re-transmission of the packet, up until all packets of data are received correctly. The data thus corrected are retransmitted to the acquisition personal computer PC, which hence provides to write the previously missing data in the recorded file.

The above-described system for the identification and recovery of transmission errors allows to recover any number of lost or incomplete data, integrating them in the data file originally received and thereby rebuilding a comprehensive file free from errors for subsequent deferred examination. As a matter of fact, in order to do this, it is simply sufficient that the number of buffer memories available on sensor S be greater than the maximum expected number of errors which may occur during data transmission in the course of a single session of electromyographic analysis.

From the preceding description it is clear how the electromyograph of the present invention has fully achieved all the set objects. In particular, with the improved electromyograph of the present invention it is possible to:

integrate in a single device, through RF sensors, information on muscle electromyographic signals with one or more pieces of functional information concerning the patient's motor activity, so as to be able to create an exact correlation between the individual steps of each specific movement or action by the patient and the corresponding muscle activity, without causing any impediment to the patient's movements;

have sealed sensors, wherein no access compartments to the inner components are provided, such as the power supply battery, nor external terminals of the battery, thereby guaranteeing a high standard of safety of use, ease of handling and of sterilisation with disinfectant liquids;

have a high standard of endurance of the sensors thanks to an efficient control system of their operational state and to use a handy, effective and simple docking device apt to house and recharge the same, when not in use, completely free from disposal problems of exhaust batteries or of deterioration/oxidation of electrical contacts, thereby positively meeting the requirements of continuous work of a hospital environment;

use simplified sensors, free from the third reference electrode, hence allowing a very remarkable decrease of patient preparation time and hence increased efficiency of use of the tool.

have electromyographic data available in real time, not corrected by transmission errors, with a short delay, which is the same for all and constant over time with respect to the instant in which the data are taken from the patient and to have, immediately after the end of the acquisition session, correct and comprehensive data, free from transmission errors, for later analysis by the physician.

The present invention has been described with particular reference to a preferred employment thereof in the field of electromyography of moving subjects, but it is clear that, other than these, many others may be the medical applications for the detection of low-intensity electrical body signals, for example in the fields of electrocardiography and electroencephalography, all within the reach of a person skilled in the field and hence falling within the scope of the present invention, which hence appears limited exclusively by the definitions in the accompanying claims.

1) Apparatus for the detection of bio-electric signals in a moving subject comprising:
a. one or more signal sensors (S) applied to the subject, each comprising means (1) for the analogical picking up of bio-electric signals, an amplifier (A) of the detected analogical signals, an analogical/digital converter (A/D) of the amplified signals, a low-consumption microcontroller (2) with independent power supply, for controlling the flow of digital signals to a radio-transmitter (3) apt to sequentially issue the digital signals to a basic unit (U);
b. one or more movement sensors (F) applied to the subject, each comprising means for the detection of a patient's motor activity in the form of an analogical electric signal of varying intensity, an amplifier (A) of the detected analogical signals, an analogical/digital converter (A/D) of the amplified signals, a low-consumption microcontroller (2) with independent power supply, for controlling the flow of digital signals to a radio-transmitter (3) apt to sequentially send the digital signals to said basic unit (U);
c. at least a basic unit (U), comprising a radio-receiver capable of receiving the signals coming from said signal sensors (S) and from said movement sensors (F), a digital/analogical converter (D/A), a microprocessor (MCU) and a USB-standard, serial interface unit, said basic unit being capable of transmitting said signals, in digital and/or analogical format, to external devices for signal interpretation and display.

2) Apparatus as in claim 1), wherein each sensor (S, F) consists of a closed and sealed element, containing the processing components of the detected signal and a means for the independent power supply of the microcontroller (2), whereto said means for the picking up of the bio-electrical signals or said means for the detection of a motor activity are electrically and mechanically connected.

3) Apparatus as in claim 2), wherein said means for the independent power supply of the microcontroller (2) consists of a rechargeable battery (5), permanently enclosed in said closed and sealed element.

4) Apparatus as in claim 3), wherein said rechargeable battery (5) does not exhibit terminals for electrical connection on the outside of said sensor (S, F).

5) Apparatus as in claim 4), further comprising a docking (D) apt to house and recharge the sensors (S, F), provided with a plurality of housings (6) for the closed and sealed element of said sensors, each housing (6) incorporating an oscillating circuit (7) for the inductive transfer of electric power to a corresponding oscillating circuit formed inside the closed and sealed element of said sensors (S, F), this last circuit providing to the recharging of the battery (5) through a rectifier.

6) Apparatus as in claim 2), wherein said picking-up means consist of disposable electrodes (1) removably fastened to corresponding electrode-carriers connected through flexible cables to the closed and sealed element of the sensor (S).

7) Apparatus as in claim 6), wherein said picking-up means consist of two electrodes (1) for picking up the useful signal and of a third equalisation electrode (EQ).

8) Apparatus as claimed in claim 6), wherein the sensor (S) is completely insulated from the environment and is further miniaturised so as to make the difference between the common-mode voltages existing on the electrodes (1) and on the amplifier (A) negligible.

9) Apparatus as in claim 8), wherein said picking-up means consist of the two signal-detection electrodes only, the sensor (S) hence lacking a third equalisation electrode.

10) Apparatus as in claim 9), further comprising two same-value additional resistances arranged, in parallel to the conductors carrying the electromyographic signal, between said signal-detection electrodes (1) and the unipotential node (B) of the differential amplifier (A).

11) Apparatus as in claim 10), wherein said additional resistances are of a sufficiently high value as not to interfere with the electromyographic signal, but significantly lower than the input resistances (Rin) of the amplifier (A).

12) Apparatus as in claim 11), wherein said additional resistances have a lower value than the input resistances (Rin) of the amplifier (A) by at least a 10⁻³ factor.

13) Apparatus as in claim 2), wherein said picking-up means consist of fixed electrodes integral with the closed and sealed element of the sensor (S).

14) Apparatus as in claim 1), wherein said movement sensors (F) are chosen in the group consisting of: accelerometers, electrogoniometers, contact switches, load cells, pressure gauges, temperature gauges.

15) Apparatus as in claim 1), wherein said sensors (S, F) further comprise a radio-receiver capable of receiving digital signals from said basic unit (U).

16) Apparatus as in claim 15), wherein said basic unit (U) comprises a radio-transmitter capable of sending digital signals to the sensors (S, F) for the control and configuration of such sensors.

17) Apparatus as in claim 3), wherein the power consumption of the battery (5) is controlled by the basic unit (U), through the configuration of said sensors (S, F) into four different activation states.

18) Apparatus as in claim 17), wherein said different activation states comprise:
a. a low-consumption SLEEP state, wherein the sensors (S, F) are active to the reception of signals coming from the basic unit (U) only for a short time window of a cycle having a predefined duration;
b. a transient state SYNC for the synchronisation of the sensors (S, F), wherein the sensor is activated for a sufficiently long reception time to allow the exchange of synchronisation data with the basic unit (U);
c. a READY waiting state, wherein all the functions of the sensor (S, F) are activated with a reduced frequency, except those concerning picking-up means or detection means;
d. an OPERATE state for data acquisition, during which the sensor (S, F) is fully active, under the control of basic unit U at the maximum operating frequency.

19) Apparatus as in claim 18), wherein the duration of the SLEEP cycle ranges between 10 and 180 seconds and the duration of the reception time window ranges between 1 and 18 msec.

20) Apparatus as in claim 18), wherein the duration of the SYNC cycle ranges between 0 and 1 second.

21) Apparatus as in claim 18), wherein the cycle frequency in the READY state is comprised in the range from 0 to about 1/100 of the maximum operating frequency.

22) Apparatus as in claim 18), wherein the maximum operating frequency is comprised in the range from about 100 to 10,000 samples/sec.

23) Apparatus as in claim 16), wherein each electrode (S, F) further comprises a plurality of buffer memories (BM), used sequentially for storing the packet of data issued to basic unit U for which basic unit U has not sent a correct reception signal to the sensor (S, F).

24) Apparatus as in claim 23), wherein the packets of data stored in such buffer memories (BM) are transmitted back to the basic unit (U) up until correct reception of the same.

25) Apparatus as in claim 24), wherein the data detected by the sensors (S, F) are delivered in real time, without correction of any transmission errors, with a short delay, which is the same for all data and constant over time with respect to the instant in which the data are taken from the patient.

26) Apparatus as in claim 24), wherein the data detected by the sensors (S, F) are delivered after full correction of any transmission errors, once the acquisition session of the patient's data has been completed.

27) Apparatus as in claim 1), wherein said external devices for the interpretation and display of the signals comprise a processor and a screen.

28) Apparatus as in claim 1), wherein said bio-electrical signals are electromyographic, electrocardiographic or electroencephalographic signals.

29) Apparatus as in claim 1), wherein said subject is a human or other animal.

30) Apparatus as in claim 2), wherein said movement sensors (F) are chosen in the group consisting of: accelerometers, electrogoniometers, contact switches, load cells, pressure gauges, temperature gauges.

31) Apparatus as in claim 2), wherein said sensors (S, F) further comprise a radio-receiver capable of receiving digital signals from said basic unit (U).
